(19) United States
(12) Patent Application Publication
(10) Pub. No.: US 2026/0203971 A1
(43) Pub. Date: Jul. 16, 2026

(54) FONT CALCULATOR FOR BURNING CONTENT ON A DOCUMENT

(71) Applicant: Hyland Software, Inc., Westlake, OH (US)

(72) Inventor: Nicholas Grace, Lakewood, OH (US)

(21) Appl. No.: 19/024,821

(22) Filed: Jan. 16, 2025

(51) Int. Cl. G06T 11/60; G06F 40/109; G16H 10/60

(52) U.S. Cl. CPC G06T 11/60; G06F 40/109; G16H 10/60

(57) ABSTRACT

A device, system or method of a healthcare enterprise can operate to generate a customized text on a virtual note and a medical document or image for burning or imprinting the customized text to the document or image. The document or image can be associated with a personal or patient identification in a display screen. The device, for example, can then operate a set of processes to burn or permanently imprint the customized text on the virtual note onto the medical document, or image, by eliminating more than one virtual layer in the display screen, based on a first set of parameters of the customized text and a second set of parameters of the medical document, the image, or the display screen so that the font of the customized text in the document or image of the display screen is acceptable.

### FIELD

This disclosure relates to computerized devices and systems, in particular within healthcare fields for burning content with a font on a document.

### BACKGROUND

Computerized networks and data management systems can include a variety of systems, devices, and technologies to enable users to create, store, access, and distribute information. Such networks can include one or more wired networks, wireless networks, or a combination thereof. Each network can include a broad range of interconnected devices, each comprising hardware, software, virtualization technology, etc., which enables the devices to send, receive, process, or store information. Examples of such devices can include mobile user devices (e.g., cell phones, tablet computers, laptop computers, etc.) stationary devices (e.g., desktop computer, servers, etc.), and network components and devices (e.g., network hubs, routers, base stations, satellite systems, etc.).

Various healthcare industries, businesses or other personal users can run into formatting issues when a traditional viewing screen is used to view content transferred from one onto another. Either the scaling process is non-existent or fails to scale the content of one standard format and viewing screen to another of different dimension or format. In a similar example, a web page viewed in a mobile browser may become distorted, too small, or much larger than expected, leading to a poor quality of experience. When such issues are encountered in a healthcare setting for transferring content of one document or form onto another on a different device, mistakes can increase, leading to a lower quality of experience or even a mistake made within the healthcare industry or other vital service. At worse, a loss of life may occur, and at best an inconvenience results. Therefore, a demand for ensuring proper scaling among layers of different documents, formats, devices and viewing screens in a virtual environment is vital.

Integrated care continues to emerge as an appropriate service delivery model to provide a safer and higher-quality patient experience. The traditional hierarchical and soloed approach, driven by the missions of single organization or department objectives should continue to evolve toward integrated pathways in the healthcare enterprise. Useful information sharing can improve the quality of care and experience. From a healthcare information systems point of view, new approaches to data aggregation, storage and sharing is needed. Healthcare enterprises will need to look for solutions based on interoperability operations tested on patient profiles and developed by Integrating the Healthcare Enterprise (IHE), other enterprise, systems or devices in particular.

### BRIEF DESCRIPTION OF THE DRAWINGS

The present disclosure will be readily understood and enabled by the detailed description and accompanying figures of the drawings. Like reference numerals can designate like features and structural elements. Figures and corresponding descriptions are provided as non-limiting examples of aspects, implementations, etc., of the present disclosure, and references to “an” or “one” aspect, implementation, etc., does not necessarily refer to the same aspect, implementation, etc., and can mean at least one, one or more, etc.

FIG. 1 is a diagram of an example overview according to one or more implementations (aspects) described herein.

FIG. 2 is an example process flow for burning text or content to another document according to one or more aspects or examples described herein.

FIG. 3 is another example process flow for burning text or content to another document according to one or more aspects or examples described herein.

FIG. 4 is another example process flow for burning text or content to another document according to one or more aspects or examples described herein.

FIG. 5 is a diagram of example components of a device that can be used within environments or implementations (aspects) herein.

FIG. 6 is another diagram of example components of a device that can be used within environments or implementations (aspects) herein.

### DETAILED DESCRIPTION

The following detailed description refers to the accompanying drawings. Like reference numbers in different drawings can identify the same or similar features, elements, operations, etc. Additionally, the present disclosure is not limited to the following description as other implementations can be utilized, and structural or logical changes made, without departing from the scope of the present disclosure.

As utilized in this disclosure, terms “component,” “system,” “interface,” and the like are intended to refer to a computer-related entity, hardware, software (e.g., in execution), and/or firmware. For example, a component can be a processor (e.g., a microprocessor, a controller, or other processing circuitry or device), a process running on a processor, a controller, an object, an executable, a program, a storage device, a computer, a tablet PC and/or a user equipment (e.g., mobile phone, etc.) with a processing device. By way of illustration, an application running on a server and the server can also be a component. One or more components can reside within a process, and a component can be localized on one computer and/or distributed between two or more computers. The term “circuitry” may refer to, be part of, or include an Application Specific Integrated Circuit (ASIC), an electronic circuit, a processor (shared, dedicated, or group), or associated memory (shared, dedicated, or group) operably coupled to the circuitry that execute one or more software or firmware programs, a combinational logic circuit, or other suitable hardware components that provide the described functionality. In some embodiments, the circuitry may be implemented in, or functions associated with the circuitry may be implemented by, one or more software or firmware modules. In some aspects, circuitry may include logic, at least partially operable in hardware.

As display technology has progressed, display screens have an increasing number of pixels into each unit of physical space, which has resulted in the pixels per inch (PPI) or the dots per inch (DPI) of display screen being higher than they have historically been, and vary from among one another. Legacy desktop user interface (UI) frameworks have built-in assumptions that the display DPI will not change during the lifetime of the process. However, the display DPIs can change several times throughout an application process's lifetime through the viewing configuration settings, for example. Some scenarios where the display scale factor/DPI changes can be during multiple-monitor setups, where each display has a different scale factor and the application is moved from one display to another (such as a 4K and a 1080p display). Such changes can occur also from docking and undocking a high DPI laptop with a low-DPI external display (or vice versa), connecting via remote desktop from a high DPI laptop/tablet to a low-DPI device (or vice versa), making a display-scale-factor settings change while applications are running. In these scenarios, applications (e.g., a windows platform or the like) can redraw or re-configure themselves for the new DPI automatically. In contrast, desktop applications do not necessarily re-configure automatically, and thus, desktop applications that do not respond to DPI or PPI changes may appear blurry or incorrectly-sized to the user. Therefore, in particular applications, aspects herein address burning operations and seek to address these issues when burning, or permanent imprinting is being performed among documents or different layers of virtual text through various scaling processes.

When a user creates a customized text or image such as a form or note that is electronic or virtual with sensitive or confidential content or information (e.g., a physician note with a signature, other note or record of a patient's medical encounter with the physician or healthcare enterprise), the content can sometimes be layered upon a document, and then permanently imprinted, or burned, onto the document with a burning or permanent imprinting operation.

For example, the content of the note (e.g., virtual content, customized text, a virtual note or virtual image) can be located or re-located so that the customized text on the virtual note is layered over at least a portion of the document for burning onto. The location on which to burn the text or note can be drawn, or placed, over the document or form the user would like to burn the text to. Alternatively, the portion of the document can be selected by moving or re-locating the text or note over a particular portion of the document to indicate where the customized text or form is to be burned onto the document, for example. This document can be in a same screen display or device, or a different display configuration, display screen, view, or different format than the creation of the note. The note can be generated in a same device or display as the document being burned to, or a different device or display. The note or content of the note can include text, symbols, images or other characters with a particular font and font size. The document could have been created in a different virtual/digital format, or created with different formatting, screen/viewing dimensions, view (display) settings or configurations than the note, which can cause inconsistencies in the content (e.g., text, image, symbols or other content) when both documents of information are combined by an imprint or burn operation.

For example, the note can include, or be, a customized text that a physician may have recorded in a medical note having content (e.g., text, characters, symbols, or other content) with a width, height or other dimension of a font or font size, but when imprinted onto a medical document, or other form, appear distorted, either too small to be easily readable, or too large resulting in other vital information of the document being covered over, or illegible, for example. For example, the document being burned to, the note or both can be associated with a personal or patient ID, which could result in being covered over by the burn operation without a proper scaling process.

A note can be generated in a same or different display or device as the document to be burned to. The note can be generated as electronic or virtual content based on a touch screen, a mouse, stylus or other application, with or without any corresponding source file. A form can also refer to electronic or virtual content. The content can include text, print, electronic handwriting, an image, or other virtual content of a note, a form, a file or a document as a virtual layer that can be imported, overlaid, imprinted, burned or viewed over another, form or document. A document can be an electronic document or file in a different or same format as the note, and have different viewing configurations or viewing setting, or different display screen dimensions having different viewing dimensions than the note. For example, the viewing configurations for the document can comprise a standard typography constant (e.g., 72 pixels per inch (PPI) or dots per inch (DPI)), or a desktop publishing point of an inch (DesktopPublishingPointOfAnInch), that is greater than a standard PPI or DPI, such as greater than 72 PPI or 72 DPI, while the note can have viewing configurations or view settings set to the standard typography constant or desktop publishing point of an inch (e.g., 72 PPI or DPI).

The note can be a first virtual layer created on a different system, device or application, or otherwise created with different operating system viewing configurations or view settings than the document as a second virtual layer. In such a case, the scaling process, or the lack of a scaling process in a burn operation could result in the content of the note being larger or smaller than the document or the portion of the document that the note is being burned to, especially when permanently imprinting or burning one layer (e.g., the first virtual layer) onto the other layer (e.g., the document). When this results in the font size being much larger, for example, the result may be that an operation of sliding the form over to the left or right is needed in order to read the full content, or a covering up of other vital patent information on the document being burned to may occur. This can lead to a poor user experience and inadequate care, especially where patient care is urgent. Even worse, vital patient information on the document could be overwritten or hidden, or the content of the form be too small to be easily readable on the document, for example.

In an aspect, a customized text can be burned onto a document or image by eliminating the virtual layers so that only one layer remains, such as when burning text or content of a virtual note (note) onto a document.

In another aspect, the burning process can include scaling a base font size of the customized text by computing a scaled base font size based on sets of parameters. The font size of the customized text or content of the note can be determined by screen viewing configurations or viewable settings of the note, or received by user input for the burning operation. The device, or system, with a processor or processing circuitry can then calculate a final font height based on the scaled base font size, and then the customized text or note can be burned or imprinted onto the document based on the final font height, for example.

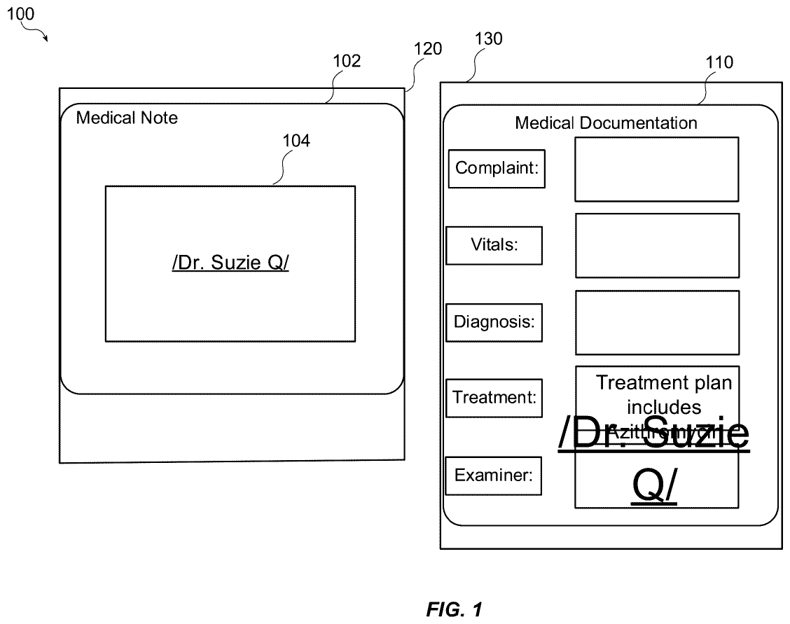

FIG. 1 illustrates an example of different virtual layers 100, which can be different from one another in various ways. For example, a first virtual layer 102 can be a note, such as a physician note, a medical note, or other virtual or digital form with customized text 104 or other content, while another second virtual layer 110 can be a medical form or document with different text, on which the first virtual layer 102 is being burned or permanently imprinted to in part or in whole. The first virtual layer 102 and the second virtual layer 110 can be documents that can include files, images, scans, pages, text, symbols, or other virtual, digital or electronic content generated by or viewed in an application of a computer. The first virtual layer 102 and second virtual layer 110 can be different from one another in this manner. The first virtual layer 102 with customized text 104 could have been created in different viewing or display screen configuration settings (or parameters) that differ from that of the second virtual layer 110, or can be generated on a same display screen or device, for example.

The first and second virtual layers 102 and 110 can be generated also on different screen displays, partially illustrated as display screens 120 and 130, respectively, with different viewing or display parameters or settings. For example, the display screen 120 can be different dimensions of height, width, or length than the display screen 130, as well as have a different PPI or DPI set for viewing. Alternatively, they could have the same dimensions, but set to different PPIs or DPIs, which may have different system viewing configurations from the operating system or application used to generate the documents, or from different screens with different screen DPIs or PPIs.

In an aspect, the display screens 120 and 130 can have different PPIs for viewing or different DPIs with respect to printable content. The first virtual layer 102, for example, could be generated or displayed in the display screen 120 with a screen PPI or DPI (e.g., 72 PPI), while the second virtual layer 110 can be generated or displayed in display screen 130 comprising a screen PPI or DPI that is greater (or less) than the screen PPI or DPI of display screen 120. As a consequence, burning or permanently imprinting one document to the other can cause a distortion effect, in which the customized text 104 of the first virtual layer 102 becomes distorted, either too large or too small, relative to the rest of the content of the virtual layer 110 that the first is being burned or permanently imprinted to, for example.

In this example, burning the customized text 104 of virtual layer 102, or the entire virtual layer 102 itself, onto virtual layer 110, or a particular portion of virtual layer 110 that is selected/overlaid with the customized text or virtual layer 102, generates a single layer of content with the customized text 104 that is too large (e.g., the signature “Dr. Suzie Q”), covering other vital information (e.g., a patient treatment, diagnosis, vital signs, complaint info, etc.) unintentionally; and thus, rendering at least a part of the medical documentation, for example, in virtual layer 110, practically illegible or unusable.

FIG. 1 illustrates the virtual layer 110 as a document with medical documentation along with the customized text 104 of the medical note of virtual layer 102 burned or permanently imprinted onto a portion of virtual layer 110. Unfortunately, the customized text 104, after being burned into the virtual layer 110, for example, covers other unintended portions of the virtual layer 110, rather than being scaled properly or according to the same parameters as the customized text in the virtual layer 102.

The customized text 104 can be a signature, for example, other content, or other text. Although illustrated as an electronic signature, for example, the customized text 104 can be other content including a virtual penned handwritten or typed text, image, or other electronic content with other characters, symbols, images, etc. For example, the customized text 104 could be any text, with characters that are alphabetic, alpha-numeric or numeric, which are in a digital/virtual format, or electronically written as with a virtual penned handwriting by an electronic pen, stylus, finger or other object being sensed by a sensor in the screen or other component (e.g., a stylus or pen).

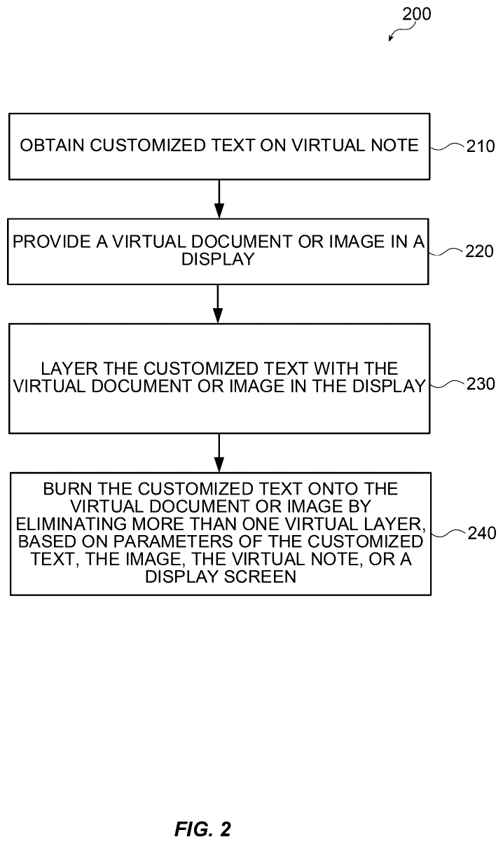

FIG. 2 illustrates an example process flow 200 for a font calculator, device (e.g., a medical device or computer device), or other component of a computing device that burns or permanently imprints text or other content on a document from another by making two different virtual layers one and for overcoming the issues discussed herein.

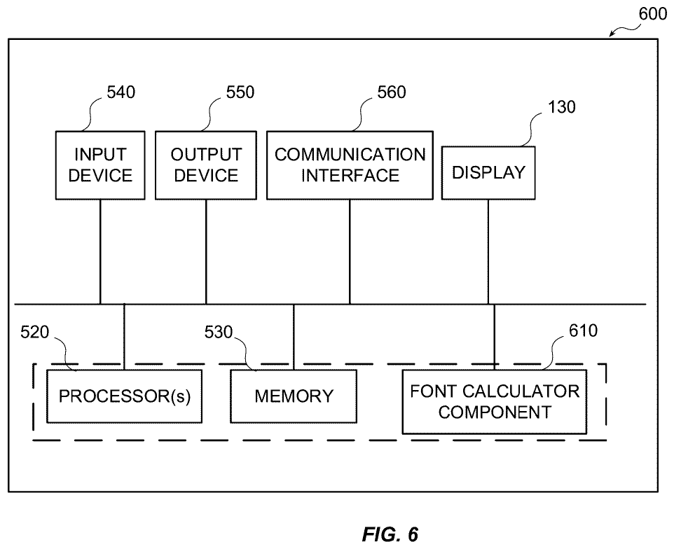

A font calculator component (e.g., font calculator component 610 of FIG. 6) of a device with a display screen can operate to burn or imprint customized text from one form to another and remedy any configuration or viewing parameters differences between the two forms, regardless of the parameters of the customized text, the form or document of the customized text, the display screen in which the customized text was generated on the form or document, the display screen of the form or document to which the customized text is being burned to, the form/document to which the customized text is being burned to, or the parameters of the portion of the form/document that the customized text is being burned to. In an aspect, the content of the customized text 104 or the note of first virtual layer 102 can be located or re-located so that the customized text on the virtual note is layered over at least a portion of the form for burning onto. The location position can be moved over a particular portion of the document to indicate where the customized text of the form is to be burned onto the other layer or second form or document, for example.

Process flow 200 can initiate at 210 with obtaining, either by generating or receiving from another device, a customized text 104 on virtual layer 102. The virtual note can be a first form or a first document upon which the customized text is generated, such as a physician's note, other document or file with content such as textual content or otherwise.

At 220, the process flow 200 includes providing or generating a second form or a second document (second form) 110 in a display (e.g., display screen 130). The display screen 120 can be a different display screen of a same or a different device that generated the first virtual layer 102. For example, the first virtual layer 102 can be generated with a device in a display screen 120 having different dimensions in height, width or length, different viewing configurations or view settings, or viewing (display screen) parameters, such as a different desktop PPI/DPI or screen PPI for its display screen compared to the display screen 120 that the second virtual layer 110 is provided in or displayed for being burned onto.

The first virtual layer 102, the second virtual layer 110, or both the first and second can be associated with a particular identification (ID), such as a patient ID, personal ID or form ID of a healthcare enterprise. Alternatively, the first or second form can have a tag or other identifier matching the two with one another, or have no identification, where the two forms are generic and not specific to any particular individual or patient.

At 230, the process flow 200 can include layering the customized text 104 on the virtual note of virtual layer 102 with the virtual medical document, or the virtual medical image, in the display screen 130. Here, virtual layer 102 of FIG. 1 can be a not (or virtual note) and the second virtual layer 110 can be a document or file in the display screen 130 of device that is providing the virtual layer 110, with the virtual layer 102/customized text 104. The virtual layer 102 can have different screen or device viewing configurations, settings or parameters when generated than the first virtual layer 110.

At 240, the process flow 200 can include burning, inserting or permanently imprinting the customized text 104 of the note based on set(s) of parameters, viewing configurations, or view settings. These parameters, viewing configurations or settings can be associated with the display screen 120, the display screen 130, the customized text 104, the virtual layer 102 with the customized text 104, the document or an image of the second virtual layer 110. Additionally, burning of the customized text 104 or virtual layer 102 onto the second virtual layer 110 can include eliminating multiple layers, including the first virtual layer 102 and the second virtual layer 110 so that only one virtual layer remains after the customized text 104 or virtual layer 102 is permanently imprinted or burned into the document of second virtual layer 110. This can result in a different form, document or file, or an updated version of the second virtual layer 110 with the customized text 104 or note. In this manner, the virtual layer 102 with customized text 104 no longer exists or is no longer present in the display screen 130, for example. Accordingly, burning or permanently imprinting is not the same as transferring the text by copying and pasting, but is based on integrating the virtual layer 102 with the customized text 104 into the virtual layer 110 so that the customized text 104 as electronically or virtually generated by type or digital writing is a part of or permanently integrated into the virtual layer 110 as a second version of the second document (second form) of virtual layer 110.

In an aspect, at 240 the process flow 200 can include, for example, burning the customized text 104 of the virtual layer 102, or the entire virtual layer 102 itself, onto the virtual layer 110 of FIG. 1 based on a scaled base font height or size, as well as a final font height or size of any dimensions or parameters of the text 104 or virtual layer 102. Height is not necessarily the parameters used, but a width, height or length of a font could be part of the height being calculated. In one example, height is being calculated and derived in the calculation.

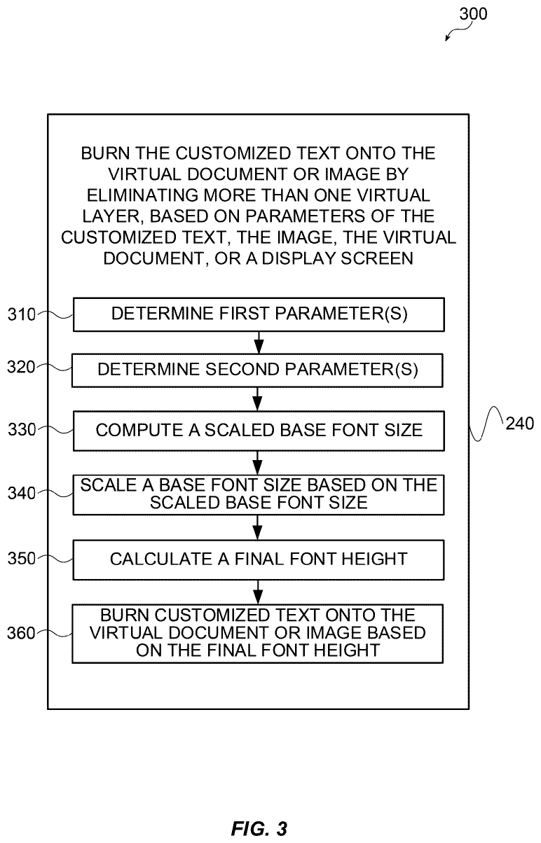

FIG. 3 illustrates another example process flow 300 of a font calculator, device (e.g., a medical device or computer device), or component of a computing device for burning text or other content on a document or form (e.g., of virtual layer 110). The process flow 300 illustrates various aspects of act 240 of FIG. 2 for burning or imprinting in a burning operation.

At 310, the process flow 300 can include determining a first set of parameters associated with the customized text 104 of the document or virtual layer 102.

The customized text 104 or content of virtual layer 102 can be any content that includes text, but is not limited to text. The content of the customized text 104 can be information of a Digital Imaging and Communication in Medicine (DICOM) content or non-DICOM content, such as, for example, information related to a DICOM image data including dimensions, size, modality used to create the data, bit depth, and settings of the medical imaging equipment used to capture a DICOM image. Other content such as Cross-Enterprise Document Sharing (XDS) content or content of another format that provides patient information (e.g., a patient ID or other patient related content), including information such as a patient's medical history, file, demographics, immunization status, radiology images, medical allergies, basic patient information (e.g., age, weight, or other patient related information), vital signs, billing information, etc. Alternatively, or additionally, other non-DICOM content can include medical image data objects such as, for example, diagnostic objects having standard object formats such as, JPEG, PDF, MPEG, TIFF, WAV, but that are not DICOM objects. Such content may also be objects having no standard information format or model so that the associated data format does not specify required or standard identifying information that is associated with the content. The customized text 104 can be associated content that refers to an XDS content, a DICOM content or content of other formats (e.g., HL7, plain text, or other data format) including a medical record or file, which can include a header and a body arranged according to a format specified by DICOM, XDS or other standards. The content or customized text 104 can include, for example, the imaging information of the corresponding X-ray, MRI, etc., which can also include pixel data or image data as a part of DICOM metadata.

The first set of parameters at 310 can include parameters of the customized text 104 that can include a base font size (or customized text base font size), such as a customized base font height, or other dimension of the customized text when originally generated on a first device or in a first display screen 120 or dimensions of the virtual note or virtual layer 102 as a virtual note base font size. The virtual note base font size can be a parameter regarding other information on the virtual note as virtual layer 102, such as, for example, the descripting information “Medical Note” in FIG. 1, or other information that may also be burned into the second document or virtual layer 110 on another device or display screen 130 that is different in dimension, PPI or DPI of the first virtual layer 102, or with a different viewing configuration or parameters, for example. When just the customized text 104 is being burned into the virtual layer 110, parameters (e.g., a customized text base font size or other customized text font parameter) associated with the customized text 104 alone could be utilized for the burning operation. Alternatively, or additionally, parameters (e.g., a virtual note base font size or other parameter or other viewing configuration/setting of the virtual layer 102) could be utilized for the burning in act 240.

At 320, the process flow 300 determines a second set of parameters. The second set of parameters can include parameters associated with one or more of: the second virtual layer 110. The second set of parameters can include one or more of: a screen PPI of the display screen displaying the virtual layer 110, or of the screen PPI parameter used to render the second virtual layer 110, a standard publishing point of an inch, a document PPI of the virtual medical document or the virtual medical image, or a desktop publishing point of an inch. The second set of parameters can include the screen/device DPI for the device that burns the note on the document (second form) 110, while the first set of parameters can be associated with the text 104 or content of the virtual layer 102 that is being burned and scaled for burning, for example.

At 330, the process flow 300 computes a scaled base font size in order to scale a base font size of the note or virtual layer 102 (BaseFontSize) of the text 104 or virtual layer 102. The base font size can refer to a customized text base font size of the customized text 104, or of the virtual note (e.g., virtual layer 102). The base font size may include or be characterized by a text (e.g., a signature or other writing) with a font, but may also refer to images or any content or data within at least a portion of the virtual layer 102 that is displayed in the display screen 120 with or without text associated with the first set of parameters.

The scaled base font size (ScaledFontSize) can be derived from, or computed, based on the first set of parameters and the second set of parameters. For example, the scaled base font size can be determined according to a ratio (Ratio) between the screen PPI of the device or display screen 130 of the virtual layer 110 that the text 104 or virtual layer 102 is being burned to, as well as the standard publishing point of an inch (e.g., 72 or other standard). The ratio (Ratio) can be the screen PPI over the standard publishing point of an inch and represented, for example, as follows: Ratio=screen PPI/StandardPublishingPointOfAnInch, or, a screen DPI/StandardPublishingPointOfAnInch.

Alternatively, the standard publishing point of an inch can be a desktop publishing point of an inch (DesktopPublishingPointOfAnInch) where the display screen 130 may, or may not, be set in viewing configuration with the standard publishing point of an inch (e.g., 72 PPI). A desktop publishing point of an inch can be a point of smallest measure for a font size, leading (the spaced between adjacent lines of type), or other content on a page, note, or document as the virtual layer 102. The desktop publishing point of an inch may be equal to the standard publishing point of an inch as a standard font point (e.g., 72 PPI or DPI), but not necessarily.

The screen PPI or DPI can be a parameter of the display screen 130 on which the virtual layer 110 is being rendered, and on which the virtual layer 102 (or customized text 104) is being burned or permanently imprinted to. The screen PPI can be determined by the diagonal of the screen in pixels divided by the diagonal in inches, or as a viewable (screen display) configuration of the device, operating system, application being used for burning to the document (second form) 110, or the display screen 130, and generating the burning operation onto the virtual layer 110 with the text 104 or virtual layer 102.

At 340, the process flow 300 operates to scale the base font size of the customized text 104, or virtual layer 102, based on the scaled base font size. For example, the scaling can include subtracting from the base font size a ratio (Ratio) of the screen PPI and the standard publishing point of an inch as the Ratio applied to the base font size. The scaling operation can be represented, for example, as follows: scaled base font size (ScaledFontSize)=[BaseFontSize−(BaseFontSize×(Ratio−1))]=[BaseFontSize−(BaseFontSize×(FontScaleMultiplier))], where the FontScaleMultiplier is a font scale multiplier equal to the Ratio minus one, or (Ratio−1), with or without the absolute value being taken thereof.

In this way, the scaling operation subtracts from the base font size the ratio as it is applied to the base font size. The ratio is multiplied by the base font size and base font size is subtracted from the result, and then this resultant of the subtraction is subtracted from the base font size to obtain a scaled font size, as follows: [BaseFontSize−(BaseFontSize×(Ratio−1))]

In an aspect, an absolute value can be taken of the ratio minus one (Ratio−1) in the operations above for obtaining the scaled base font size and scaling the base font size, according to the font scale multiplier (FontScaleMultiplier). The font scale multiplier can be represented, for example, as follows: FontScaleMultiplier=Absolute value (abs)|((Screen PPI or DPI/StandardPublishingPointOfAnInch)−1) 51 , where the Screen PPI or Screen DPI is based or derived from the device burning the note to the document.

Alternatively, as at act 330 or other acts, the standard publishing point of an inch (StandardPublishingPointOfAnInch) can be replaced by the desktop publishing point of an inch depending on which one is used at 330 to keep calculation operations uniform, for example.

At 350, the process flow 300 continues with calculating a final base font height (FinalFontHeight) based on the scaled base font size, and then at 360 burning the customized text 104 or note 102 onto the virtual document (e.g., virtual layer 110) based on the final base font height. The final font height can be used for burning the virtual layer 102 or the customized text 104 by using the scaled font size multiplied by the document (device/screen) PPI divided by the desktop publishing point of an inch, or the document (device/screen) PPI divided by the standard publishing point of an inch. The final base font height can be represented as follows:

FinalFontHeight=ScaledFontSize×(a document or device PPI (or DPI)/DesktopPublishingPointOfAnInch). Here, the scaled font base font size is multiplied by a (document (screen) PPI or DPI) and then divided by a desktop publishing point of an inch for use in the burning operation or permanently imprinting the customized text 104 or virtual layer 102 onto the virtual layer 110. The desktop publishing point of an inch can be equal to the standard publishing point of an inch (e.g., 72 PPI or DPI), for example, depending on the display screen 130 of the virtual layer 110 that the note or virtual layer 102 or customized text 104 is being burned to.

In an example, a base font size can be equal to 12, a screen PPI of the virtual layer 110 on which the customized text 104 or virtual layer 102 is being burned to can be 96 PPI, and the standard publishing point of an inch equal to 72. In this example, the scaled base font size (or ScaledFontSize) can be determined at 330 as 12−(12 ((96/72)−1)), or, in other words, the base font size (e.g., 12) minus the following: the base font size multiplied by the ratio (96/72) subtracted by the base font size (BaseFontSize×(Ratio−1)), as represented in c) above as: BaseFontSize - (BaseFontSize×(FontScaleMultiplier)), where the FontScaleMultiplier is the ratio minus one (Ratio−1), and where the ratio can be the screen PPI (or DPI as used in this disclosure) over the standard publishing point of an inch, or the document PPI) over the standard publishing point of an inch. The screen PPI (or DPI) can be of the display screen 130 of the document (second form) 110 that the virtual layer 102 or text 104 is being burned to, while the document PPI (or DPI) be the document PPI of document (second form) 110 based on the viewing configurations it was generated, or being rendered.

In various aspects, examples, or embodiments described herein the PPI can be replaced by a DPI, especially for printable, printed or scanned documents. Thus, the screen PPI can be a device or screen DPI, and a document PPI can be a document DPI, rather than a screen PPI or a document PPI throughout this disclosure. A PPI and DPI can otherwise be used interchangeably in this disclosure, where a PPI relates to viewing a screen and the DPI relates to printing a print out. For example, the final base font height (or, FinalFontHeight) can be configured according to a ScaledFontSize×(a document or device PPI/DesktopPublishingPointOfAnInch), or a ScaledFontSize×(a document or device DPI/DesktopPublishingPointOfAnInch).

In an aspect, depending on the display screen 130 or the viewing configurations in which the document (second form) 110 was generated, the final font height can be determined based on different variables as a function of a document PPI (or DPI) threshold, respectively. The document PPI threshold can be a predetermined threshold that the burning operations used determine which variable to use for determining the final font height from the scaled font size. If the document PPI or DPI, or device PPI or DPI, of the document 110 that is being burnt to is greater than an integer, for example, then the document PPI or DPI is used for determining the final font height for burning the virtual layer 102 or text 104 with the scaled font size as described above; otherwise, the screen PPI or screen DPI is used for computing the final font height. The predetermined threshold can be any integer (e.g., 0, or the like) so that if the document PPI or DPI is greater that the predetermined threshold, then the final font height can be determined by multiplying the scaled base font size (ScaledFontSize) by a final font height ratio of the document PPI or DPI and the desktop publishing point of an inch; otherwise the screen PPI or DPI can be used in in final font height ratio instead of the document PPI or DPI. For example, the final based font height can be determined by multiplying the scaled base font size by the final font height ratio as the screen PPI or DPI by the desktop publishing point of an inch, or the inversion thereof, when the document PPI or DPI does not satisfy the predetermined threshold.

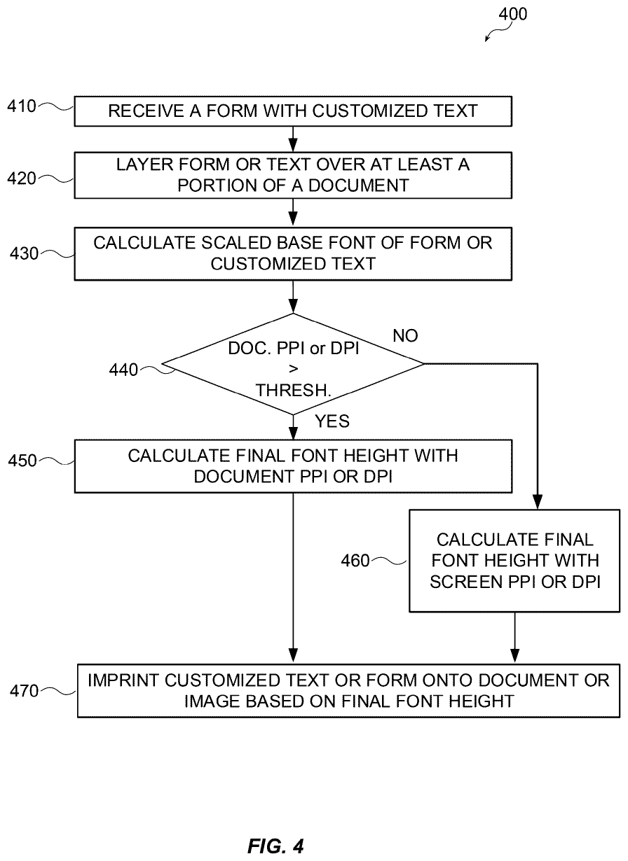

FIG. 4 illustrates another example process flow 400 for burning operations discussed herein. Acts 410 thru 430 flow similarly as in FIGS. 2 and 3 as a part of the same acts as process flows 200 and 300 or individually different. At 410, a device (e.g., a medical device, computer or other processing device) or system, with a processor or processing circuitry and memory, receive or obtain a note of a virtual layer 102 with customized text 104.

At 420, the process flow 400 includes layering or moving the virtual layer 102 or text 104 itself over at least a portion of the document (second form) 110 to be burned thereon. For example, the text 104 or virtual layer 102 can be drawn at a position, and then re-positioned, or re-located, to a portion of the document of virtual layer 110 to be burned to.

At 430, the process flow 400 includes scaling the based font of the virtual layer 102 or text 104 by calculating a scaled base font. The scaled base font can be calculated for the virtual layer 102, the text 104, or both if they are different from one another as separate operations. The scaled base font can be determined as discussed above as the scaled base font size (ScaledFontSize)=[BaseFontSizep−(BaseFontSize×(Ratio−1))]=[BaseFontSize−(BaseFontSize×(FontScaleMultiplier))], where the FontScaleMultiplier is a font scale multiplier equal to the Ratio minus one, or (Ratio−1)), with or without the absolute value being taken thereof, and Ratio is a ratio between the screen PPI or DPI of the device or display screen 130 that is burning the note and the standard publishing point of an inch (e.g., 72).

At 440, the process flow 400 makes a determination or decision for whether or not the document PPI or DPI is greater than a predetermined threshold. For example, a determination as a part of the process flow can be made as to whether the document PPI of the virtual layer 110 or device of the display screen 130 is greater than the predetermined threshold, or a document PPI or DPI threshold. If the document PPI, for example, is greater than the predetermined threshold (e.g., 0, 1 or other integer), then the document PPI or DPI can be used; otherwise, the screen/device PPI could be used, or the screen/device DPI can be used where document DPI is not greater than the predetermined threshold.

For example, the predetermined threshold can be zero to determine whether a document has a PPI or DPI (e.g., a text file), and such a non-image file may be rendered using a screen/system PPI or DPI when the document PPI or DPI is zero; otherwise an image file could be what is being burned to, and thus, the file or document's PPI or DPI be used for a final calculation step of the final font height.

The final base font height (or final font height) can be computed according to the following representation when the document PPI (or DPI) satisfies the predetermined threshold by being greater than the predetermined threshold (e.g., greater than zero) at 450 as follows:

FinalFontHeight=ScaledFontSize×(a document or device PPI (or DPI)/DesktopPublishingPointOfAnInch); otherwise, where the document PPI or DPI is determined to not satisfy the predetermined threshold (e.g., not greater than zero), the final base font height, or final font height can be computed as follows: FinalFontHeight=ScaledFontSize×(Screen/Device PPI (or DPI)/DesktopPublishingPointOfAnInch).

Here, the final font height ratio can be a Screen/Device PPI or DPI over the desktop publishing point of an inch (DesktopPublishingPointOfAnInch), when the document PPI or DPI satisfies the predetermined threshold at 460 (e.g., being equal to zero).

At 460, the process flow 400 imprints the customized text 104 or virtual layer 102 onto the document (second form) of virtual layer 110 using the final base font height. Thus, the process flow acts enable a device with any screen or device DPI or PPI to calculate the correct font height for burning the document so that there is no change from when a device has a PPI/DPI of 72, 96, 300 or other value.

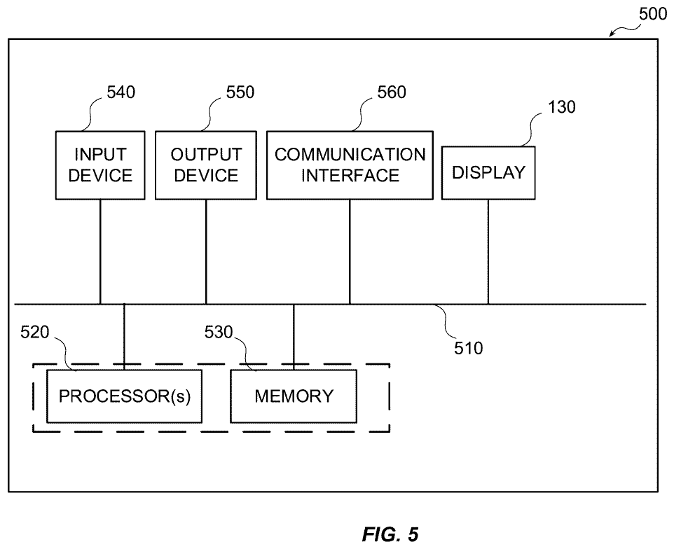

FIG. 5 is a diagram of example components of a device 500 that can be used with/within environment or systems described in FIG. 1 thru 4 for computing a final font for burning a text onto document or other file of a second virtual layer 110. Device 500 can be a medical device, computing device, mobile device, or other networked device for receiving and burning text onto a document, such as in a medical setting or other setting. As depicted, device 500 can include bus 510, processor(s) 520, memory 530, input device 540, output device 550, communication interface 560, as well as the display screen 130. However, the precise components of device 500 can vary between implementations. For example, depending on the implementation, device 500 can include fewer components, additional components, different components, or differently arranged components than those illustrated in FIG. 5.

Bus 510 can permit communication among the components of device 500. Processor(s) 520 can include one or more processors, microprocessors, data processors, co-processors, network processors, application-specific integrated circuits (ASICs), controllers, programmable logic devices (PLDs), chipsets, field-programmable gate arrays (FPGAs), or other components that can interpret or execute instructions or data. Processor 520 can control the overall operation, or a portion thereof, of device 500, based on, for example, an operating system (not illustrated), and/or various applications. Processor 520 can access instructions from memory 530, from other components of device 500, or from a source external to device 500 (e.g., a network or another device), and be separate from the memory 530 or integrated with the memory 530 as integrated processing circuitry.

Memory 530 can include memory and/or secondary storage, and be separate or a part of or integrated in the processor(s) 520 as processing circuitry. For example, memory 530 can include random access memory (RAM), dynamic RAM (DRAM), read-only memory (ROM), programmable ROM (PROM), flash memory, or some other type of memory. Memory 530 can include a hard disk (e.g., a magnetic disk, an optical disk, a magneto-optic disk, or a solid state disk) or some other type of computer-readable medium, along with a corresponding drive. A computer-readable medium can be defined as a non-transitory memory device. A memory device can include space within a single physical memory device or spread across multiple physical memory devices.

Input device 540 can include one or more components that permit a user to input information into device 500. For example, input device 540 can include a keypad, a button, a switch, a knob, fingerprint recognition logic, retinal scan logic, a web cam, voice recognition logic, a touchpad, an input port, a microphone, a display, or some other type of input component. Output device 550 can include one or more components that permit device 500 to output information to a user. For example, output device 550 can include a display, light-emitting diodes (LEDs), an output port, a speaker, or some other type of output component.

Communication interface 560 can include one or more components that permit device 500 to communicate with other devices or networks. For example, communication interface 560 can include some type of wireless or wired interface. Communication interface 560 can also include an antenna (or a set of antennas) that permit wireless communication, such as the transmission and reception of radio frequency (RF) signals.

As described herein, device 500 can perform certain operations in response to processor 520 executing software instructions contained in a computer-readable medium, such as memory 530. The software instructions can be read into memory 530 from another computer-readable medium or from another device via communication interface 560. The software instructions contained in memory 530 can cause processor 520 to perform one or more processes described herein. Alternatively, hardwired circuitry can be used in place of, or in combination with, software instructions to implement processes described herein. Thus, implementations described herein are not limited to any specific combination of hardware circuitry and software.

The processor(s) 520 with memory 530 can be configured to perform any one or more of the aspects, examples, or embodiments described and detailed with FIG. 1 thru 4. For example, the processing circuitry or processor(s) 520 with the memory 530 can be configured to receive a virtual note with a customized text or other content in the display screen 130. The processor(s) 520 can be configured to overlay the virtual note over a medical document, or a medical image, associated with a personal or patient identification, in the display screen 130 and imprint the customized text onto the medical document, or the medical image, by integrating a plurality of layers comprising the virtual note (e.g., virtual layer 102) with the customized text (e.g., customized text 104) and the medical document (or medical image) (e.g., document (second form) of virtual layer 110), into one layer with the customized text and the medical document, or the medical image, based on parameters of the plurality of layers and of the display screen 130.

The parameters of the plurality of layers or document and of the display screen can be a customized text base font size (a base font size), a screen PPI or DPI of the display screen 130 or a display setting configuration of an application or operating system of the device 500, a document PPI of the medical document or the medical image (e.g., document (second form) of virtual layer 110), a desktop publishing point of an inch, or a standard publishing point of an inch.

The processor(s) 520 can operate to imprint the customized text or other content onto the medical document, or the medical image, for example, by determining whether the document PPI or DPI satisfies a predetermined threshold. In response to the document PPI or DPI satisfying the predetermined threshold, the processor(s) 520 can derive a final font height for imprinting or burning the customized text onto the medical document, or the medical image, based on the document PPI or DPI. In response to the document PPI not satisfying the predetermined PPI/DPI threshold, the processor(s) 520 can operate to derive the final font height for burning the customized text onto the medical document, or the medical image, based on the screen PPI or DPI of the display screen 130 or a device PPI or DPI of the device 500. The device PPI or DPI can be determined based on an viewing configuration (e.g., a PPI/DPI awareness setting), a screen configuration, a primary connected monitor at the time of use or sign in of an application or a font calculator component (e.g., font calculator component 610 of FIG. 6) of the application or device burning the note to the document, for example. The screen PPI or DPI can be determined from the screen displaying the document 110 or a viewing configuration or setting for the document 110. The document PPI or DPI can be determined from the document 110 or a viewing configuration or setting of the document 110 or an application rendering the document 110.

The processor(s) 520, for example, can scale a base font size of the customized text on the virtual note by computing a scaled base font size, and then calculate a final font height based on the scaled base font size in order to use the final font height to imprint the customized text onto the medical document, or the medical image and generate the single layer or document with a uniform content or text between the two virtual layers (e.g., a first virtual layer 102 and second virtual layer 110).

The processor(s) 520 can calculate the final font height based on the parameters of the plurality of layers (e.g., the note of virtual layer 102 and document (second form) of virtual layer 110) and the display screen 130 or a display setting for the display screen 130 or the device 500. The final font height, for example, by calculating a final font height based on the parameters of the plurality of layers and the display screen, wherein the final font height equals a scaled font size multiplied by a ratio of document pixels per inch (PPI)/dots per inch (DPI), or a screen PPI/DPI, and a desktop publishing point of an inch.

FIG. 6 illustrates another example of device 600 with components of device 500 with a font calculator component 610 that can be a part of or integrated in the processor(s) 520 with memory 530 as different components. The font calculator component 610 can be configured to determine a final font height for burning the content of one document to another document of display screen 130, which were created or generated with different display settings or in different display screens with different settings. For purposes of scaling the base font size of the text or note of virtual layer 102, the font calculator component can determine a base font size of the text 104 or note of the first virtual layer 102 and scale this by a scaled font size by multiplying the base font size by one minus a ratio minus one such as by: [BaseFontSize−(BaseFontSize×(ratio−1))]=[BaseFontSize−(BaseFontSize×(FontScaleMultiplier))], where the FontScaleMultiplier is a font scale multiplier equal to the Ratio minus one, or the absolute value of (Ratio−1)) and Ratio can be a ratio of the screen/device PPI or screen/device DPI over a standard publishing point of an inch. In other words, the base font size can be multiplied by (Ratio−1) and the resultant then subtracted from the base font size. The screen PPI or DPI can be a screen or system viewing parameter of the device or document that the content is being burned to.

The font calculator component 640 can then calculate a final font height for burning the content of document (first form) 102 to document (second form) 110 multiplying the scaled base font by a final font height ratio of a desktop publishing point of an inch over the screen PPI/DPI or a document PPI/DPI, depending on a document PPI/DPI predetermined threshold as discussed herein. The processor(s) 520 or the font calculator component 610 can then burn the content of first virtual layer 102 or the customized text 104 therein onto the document (second form) of virtual layer 110 using the final font height for the burning operation.

Examples herein can include subject matter such as a method, means for performing acts or blocks of the method, at least one machine-readable medium including executable instructions that, when performed by a machine (e.g., a processor with memory, an application-specific integrated circuit, a field programmable gate array, programmable logic device, or the like) cause the machine to perform acts of the method or of an apparatus or system for concurrent communication using multiple communication technologies according to implementations and examples described.

While the methods or process flows described within this disclosure are illustrated in and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be necessarily utilized to implement one or more aspects or embodiments of the description herein. Further, one or more of the acts depicted herein may be carried out in one or more separate acts and/or phases.

The above description of illustrated examples, implementations, aspects, etc., of the subject disclosure, including what is described in the Abstract, is not intended to be exhaustive or to limit the disclosed aspects to the precise forms disclosed. While specific examples, implementations, aspects, etc., are described herein for illustrative purposes, various modifications are possible that are considered within the scope of such examples, implementations, aspects, etc., as those skilled in the relevant art can recognize.

In this regard, while the disclosed subject matter has been described in connection with various examples, implementations, aspects, etc., and corresponding Figures, where applicable, it is to be understood that other similar aspects can be used or modifications and additions can be made to the disclosed subject matter for performing the same, similar, alternative, or substitute function of the subject matter without deviating therefrom. Therefore, the disclosed subject matter should not be limited to any single example, implementation, or aspect described herein, but rather should be construed in breadth and scope in accordance with the appended claims below.

In particular regard to the various functions performed by the above described components or structures (assemblies, devices, circuits, systems, etc.), the terms (including a reference to a “means”) used to describe such components are intended to correspond, unless otherwise indicated, to any component or structure which performs the specified function of the described component (e.g., that is functionally equivalent), even though not structurally equivalent to the disclosed structure which performs the function in the herein illustrated exemplary implementations. In addition, while a particular feature can have been disclosed with respect to only one of several implementations, such feature can be combined with one or more other features of the other implementations as can be desired and advantageous for any given application.

As used herein, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form. Furthermore, to the extent that the terms “including”, “includes”, “having”, “has”, “with”, or variants thereof are used in either the detailed description or the claims, such terms are intended to be inclusive in a manner similar to the term “comprising.” Additionally, in situations wherein one or more numbered items are discussed (e.g., a “first X”, a “second X”, etc.), in general the one or more numbered items can be distinct, or they can be the same, although in some situations the context can indicate that they are distinct or that they are the same.

It is well understood that the use of personally identifiable information should follow privacy policies and practices that are generally recognized as meeting or exceeding industry or governmental requirements for maintaining the privacy of users. In particular, personally identifiable information data should be managed and handled to minimize risks of unintentional or unauthorized access or use, and the nature of authorized use should be clearly indicated to users.

What is claimed is:

1. A non-transitory computer-readable medium of a medical device comprising:
instructions that when executed by one or more processors of a medical device, cause the one or more processors to:
generate a customized text on a virtual note;
provide a virtual medical document, or a virtual medical image, that is associated with a patient identification (ID), in a display screen;
layer the customized text on the virtual note with the virtual medical document, or the virtual medical image, in the display screen; and
burn the customized text of the virtual note onto the virtual medical document, or the virtual medical image, by eliminating more than one virtual layer in the display screen, based on a first set of parameters of the customized text and a second set of parameters of one or more of: the virtual medical document, the virtual medical image, or the display screen.

2. The non-transitory computer-readable medium of claim 1, wherein burning the customized text onto the virtual medical document, or the virtual medical image, comprises:
calculating a scaled base font height of the customized text on the virtual note based on the first set of parameters and the second set of parameters; and
burning the customized text onto the virtual medical document, or the virtual medical image, based on the scaled base font height.

3. The non-transitory computer-readable medium of claim 2, wherein burning the customized text onto the virtual medical document, or the virtual medical image, further comprises:
calculating a final font height based on the scaled base font height, a document pixels per inch (PPI) or dots per inch (DPI) of the virtual medical document or the virtual medical image, and a desktop publishing point of an inch.

4. The non-transitory computer-readable medium of claim 1, wherein the instructions further cause the one or more processors to:
determine the first set of parameters of the customized text, the first set of parameters comprising at least one of: a customized text base font size of the customized text or a virtual note base font size of the virtual note.

5. The non-transitory computer-readable medium of claim 1, wherein the instructions further cause the one or more processors to:
determine the second set of parameters associated with one or more of: the virtual medical document, the virtual medical image, or the display screen with the virtual medical document, or with the virtual medical image, the second set of parameters comprising at least one of: a screen/device PPI or DPI of the display screen, a standard publishing point of an inch, a document PPI or DPI of the virtual medical document or the virtual medical image, or a desktop publishing point of an inch.

6. The non-transitory computer-readable medium of claim 1, wherein burning the customized text onto the virtual medical document, or the virtual medical image, further comprises:
scaling a base font size of the customized text on the virtual note by computing a scaled base font size based on the first set of parameters and the second set of parameters; and
calculating a final font height based on the scaled base font size; and
burning the customized text onto the virtual medical document, or the virtual medical image, based on the final font height.

7. The non-transitory computer-readable medium of claim 1, wherein the instructions further cause the one or more processors to:
re-locate the customized text on the virtual note to be layered over a portion of the virtual medical document, or the virtual medical image, in the display screen for burning onto, wherein the customized text is larger than the portion of the virtual medical document, or the virtual medical image.

8. The non-transitory computer-readable medium of claim 7, wherein the instructions further cause the one or more processors to:
burn the customized text on the virtual note onto the virtual medical document, or the virtual medical image, based on the first set of parameters and the second set of parameters, by imprinting the customized text within the portion of the virtual medical document, or the virtual medical image, wherein the customized text burned onto the portion of the virtual medical document, or the virtual medical image is smaller than the portion of the virtual medical document, or the virtual medical image.

9. The non-transitory computer-readable medium of claim 1, wherein the display screen comprises a screen/device PPI or DPI that is larger than a standard typography constant.

10. The non-transitory computer-readable medium of claim 1, wherein the instructions further cause the one or more processors to:
determine whether to burn the customized text on the virtual note onto the virtual medical document, or the virtual medical image, using a document PPI or a screen / device PPI of the second set of parameters, based on a predetermined PPI threshold.

11. A medical device, comprising:
a memory;
a display screen; and
one or more processors configured, when executing instructions stored in the memory, to:
receive a virtual note with a customized text in the display screen;
overlay the virtual note over a medical document, or a medical image, associated with a personal identification, in the display screen;
imprint the customized text onto the medical document, or the medical image, by integrating a plurality of layers comprising the virtual note with the customized text and the medical document, or the medical image, into one layer with the customized text and the medical document, or the medical image, based on parameters of the plurality of layers and of the display screen.

12. The medical device of claim 11, wherein the parameters of the plurality of layers and of the display screen comprise: a customized text base font size, a screen PPI of the display screen, a document PPI of the medical document or the medical image, a desktop publishing point of an inch, and a standard publishing point of an inch.

13. The medical device of claim 12, wherein the one or more processors are further configured to:
imprint the customized text onto the medical document, or the medical image, by determining whether the document PPI satisfies a predetermined PPI threshold; and
in response to the document PPI satisfying the predetermined PPI threshold, derive a final font height to imprint the customized text onto the medical document, or the medical image, based on the document PPI; and
in response to the document PPI not satisfying the predetermined PPI threshold, derive the final font height to imprint the customized text onto the medical document, or the medical image, based on the screen PPI of the display screen.

14. The medical device of claim 11, wherein the one or more processors are further configured to:
scale a base font size of the customized text on the virtual note by computing a scaled base font size; and
calculate a final font height based on the scaled base font size; and
imprint the customized text onto the medical document, or the medical image, based on the final font height.

15. The medical device of claim 11, wherein the one or more processors are further configured to:
calculating a final font height based on the parameters of the plurality of layers and the display screen, wherein the final font height equals a scaled font size multiplied by a final font height ratio of document pixels per inch (PPI)/dots per inch (DPI), or a screen PPI/DPI, and a desktop publishing point of an inch.

16. The medical device of claim 11, wherein the one or more processors are further configured to:
calculating a font scale multiplier by calculating one subtracted from a ratio, the ratio comprising a screen PPI to a desktop publishing point of an inch, and calculating an absolute value of the one subtracted from the ratio; and
imprinting the customized text onto the medical document, or the medical image, based on the font scale multiplier.

17. A system of a healthcare enterprise comprising:
a display screen; and
one or more processors configured to execute instructions stored in a memory that cause the one or more processors to:
generate a customized text of a virtual note based on a user input;
provide a virtual document that is associated with a personal identification (ID), in the display screen;
layer the customized text of the virtual note over a portion of the virtual document in the display screen; and
burn the customized text of the virtual note onto the portion of the virtual document by eliminating more than one virtual layer in the display screen, based on a first set of parameters of the customized text and a second set of parameters of the virtual document and the display screen.

18. The system of claim 17, wherein the one or more processors are further configured to:
calculate a scaled base font height of the customized text on the virtual note based on the first set of parameters and the second set of parameters;
calculate a final font height based on the scaled base font height; and
burn the customized text onto the virtual document based on the final font height.

19. The system of claim 18, wherein the first set of parameters comprise a customized text base font size of the customized text or a virtual note base font size of the virtual note, and the second set of parameters comprise a screen PPI, a standard publishing point of an inch, a document pixels per inch (PPI) of the virtual document and a desktop publishing point of an inch.

20. The system of claim 18, wherein the one or more processors are further configured to:
determine whether to burn the customized text on the virtual note onto the virtual document using a document PPI or a screen PPI, based on a predetermined PPI threshold.
